(19) United States
(12) Patent Application Publication
(10) Pub. No.: US 2006/0258684 A1
(43) Pub. Date: Nov. 16, 2006

(54) 2-[2-[4-[(4-chlorophenyl) phenylmethyl]-1-piperazinyl]ethoxy]acetic acid monohydrochloride as anti-allergenic compound and process for its production

(76) Inventors: Prasad Shiva Singh, Ahmedabad (IN); Jaweed Mohammed Siddiqui Mukarram, Aurangabad (IN); Yekanathsa Aravind Merwade, Aurangabad (IN); Reyaz Anjum Khan, Aurangabad (IN)

Correspondence Address: BIO INTELLECTUAL PROPERTY SERVICES (BIO IPS) LLC, 8509 KERNON CT., LORTON, VA, 22079, US

(21) Appl. No.: 10/554,696

(22) PCT Filed: May 21, 2003
(86) PCT No.: PCT/IB03/01947

§ 371 Date: Feb. 23, 2006

(51) Int. Cl. A61K 31/495; C07D 241/04

(52) U.S. Cl. 514/255.04; 544/396

(57) ABSTRACT

An anti-allergenic compound having therapeutic value and a process for its manufacture. The disclosure is directed to 2-[2-[4-[(4-chlorophenyl)phenylmethyl]-1piperazinyl]ethoxy]acetic acid monohydrochloride, to compositions containing 2-[2-[[(4chlorophenyl)phenylmethyl]-1-piperazinyl]ethoxy]acetic acid monohydrochloride, and to a process for the preparation of 2-[2-[[(4-chlorophenyl)phenylmethyl]-1-piperazinyl]ethoxy]acetic acid monohydrochloride.

### REFERENCE TO RELATED APPLICATION

This application claims the priority of U.S. Provisional Application No. ______, filed on May 19, 2003, which is hereby incorporated by reference in its entirety.

### FIELD OF THE INVENTION

This invention is directed to an anti-allergenic compound having therapeutic value and a process for its manufacture. In particular, the present invention is directed to 2-[2-[4-[(4-chlorophenyl)phenylmethyl)-1-piperazinyl]ethoxy]acetic acid monohydrochloride, to compositions containing 2-[2-[4-[(4-chlorophenyl)phenylmethyl]-1-piperazinyl]ethoxy]acetic acid monohydrochloride, and to a process for the preparation of 2-[2-[4-[(4-chlorophenyl)phenylmethyl]-1-piperazinyl]ethoxy]acetic acid monohydrochloride.

### BACKGROUND OF THE INVENTION

Cetirizine (2-[2-[4-[(4-chlorophenyl)phenylmethyl)-1-piperazinyl]ethoxy]acetic acid) and its dihydrochloride salt are well established as drugs for the treatment of allergic syndromes, such as chronic and acute allergic rhinitis, allergic conjunctivitis, pruritus, and urticaria, etc. U.S. Pat. No. 4,525,358 discloses these compounds and the preparation of aliphatic carboxylic acids substituted with 1-alkoxy-4-alkylpiperazines having the formula shown below: 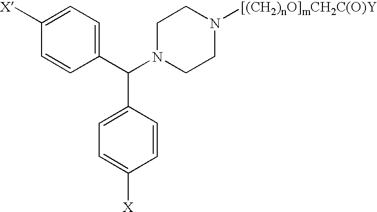
where Y is an ester, hydroxy or amino group, X and X′ are independently hydrogen, halo, linear or branched lower alkoxy or trifluoromethyl and m and n are the integers 1 or 2. A number of reaction routes for the preparation of these acetic acid derivatives are disclosed, e.g., the reaction of 1-(diphenylmethyl)-piperazine with an omega haloacetamide followed by hydrolysis, the reaction of the alkali metal salt of an omega [4-(diphenylmethyl)-1-piperazinyl]alkanol with a 2-haloacetamide followed by hydrolysis, etc. The yields as reported therein, are rather low, around 47%. Further, hydrolysis and pH correction lead to cetirizine hydrochloride.

International Patent Application PCT/HU00/00123 discloses the preparation of [2-[4-(α-phenyl-p-chlorobenzyl)piperazin-1-yl]ethoxy]acetic acid by hydrolysis of its amide or acetate derivatives. The amide and acetate derivatives of [2-[4-(α-phenyl-p-chlorobenzyl)piperazin-1-yl]ethoxy]acetic acid are prepared by the reaction of 1-[(4-chlorophenyl)phenylmethyl)piperazine with 2-chloroethoxy acetate and 2-chloroethoxy acetamide, respectively, in the presence of a metal hydride. Salts or free acids are generated after appropriate hydrolysis.

UK Patent Application No. 2,225,321 discloses that 2-[2-[4-[(4-chlorophenyl)phenylmethyl]-1-piperazinyl]ethoxy]acetic acid may be prepared by hydrolyzing 2-[4-[(4-chlorophenyl)phenylmethyl]-piperazinyl]-ethoxy)acetonitrile with base or acid. The nitrile is prepared by the reaction of racemic 1-[(4-chlorophenyl)phenylmethyl]piperazine with 2-chloroethoxyacetonitrile.

European Patent No. 058146 describes the synthesis of of 2-[2-[4-[(4-chlorophenyl)phenylmethyl]-1-piperazinyl]ethoxy]acetic acid and its dihydrochloride salt by the condensation of 1-[(4-chlorophenyl)phenylmethyl]piperazine with 2-haloacetic acid in xylene in the presence of anhydrous sodium carbonate as an acid scavenger in 54.7% yield. Conversion of cetirizine into its dihydrochloride salt is performed by hydrolyzing cetirizine amide and subsequent pH correction.

U.S. Pat. No. 6,100,400 discloses the synthesis of 2-[2-[4-[(4-chlorophenyl)phenylmethyl]-1-piperazinyl]ethoxy]acetic acid ester by reacting 1-[(4-chlorophenyl)phenylmethyl]piperazine with a haloalkyl ester in the presence of a tertiary amine solvent and an acid scavenger at a temperature of at least 100° C.

U.S. Pat. No. 6,255,487 describes the synthesis of cetirizine using amide, nitrile, alkali metal, and alkyl esters of 2-[2-[4-[(4-chlorophenyl)phenylmethyl]-1-piperazinyl]ethoxy]acetic acid as intermediates. The carboxyl derivatives of 2-[2-[4-[(4-chlorophenyl)phenylmethyl]-1-piperazinyl]ethoxy]acetic acid, on suitable acid or base hydrolysis, yield cetirizine.

Cetirizine is widely used as the active ingredient of antiallergic pharmaceutical compositions. However, newer therapeutically active derivatives of cetirizine and newer, cheaper, easier to perform and high yielding processes for its preparation are needed. The present invention relates to a novel process for the preparation of 2-[2-[4-[(4-chlorophenyl)phenylmethyl]-1-piperazinyl]ethoxy]acetic acid and its monohydrochloride salt.

### SUMMARY OF THE INVENTION

The present invention is directed to an anti-allergenic compound having therapeutic value and a process for its manufacture.

In a first embodiment, the invention is directed to 2-[2-[4-[(4-chlorophenyl)phenylmethyl]-1-piperazinyl]ethoxy]acetic acid monohydrochloride of Formula (I).

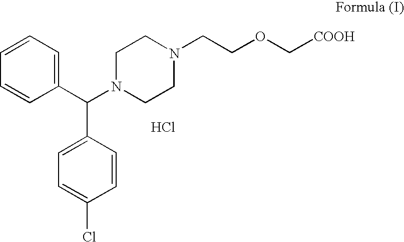

In a second embodiment, the invention is directed to a process for the preparation of the compound of Formula (I), comprising reacting 4-chlorobenzhydryl piperazine with 2-chloroethanol to form 2-[4-[(4-chlorophenyl)phenylmethyl]-1-piperazinyl]ethanol, converting that product to 2-[2-[4-(4-chlorophenyl)phenylmethyl)-1-piperazinyl]ethoxy]acetic acid and converting the latter to 2-[2-[4-(4-chlorophenyl)phenylmethyl]-1-piperazinyl]ethoxy]acetic acid monohydrochloride.

In a third embodiment, the invention is directed to pharmaceutical compositions containing 2-[2-[4-((4-chlorophenyl)phenylmethyl]-1-piperazinyl]ethoxy]acetic acid monohydrochloride.

### BRIEF DESCRIPTION OF THE DRAWINGS

FIG. 1 is a Differential Scanning Calorimetry (DSC) thermogram of 2-[2-[4-[(4-chlorophenyl)phenylmethyl)-1-piperazinyl]ethoxy)acetic acid monohydrochloride.

FIG. 2 is a ¹³C Nuclear Magnetic Resonance (NMR) spectrum in d₆-DMSO of 2-[2-[4-[(4-chlorophenyl)phenylmethyl]-1-piperazinyl]ethoxy]acetic acid monohydrochloride.

FIG. 3 is an X-Ray Diffraction Pattern (XRD) of 2-[2-[4-[(4-chlorophenyl)phenylmethyl]-1-piperazinyl]ethoxy]acetic acid monohydrochloride.

FIG. 4 is a Differential Scanning Calorimetry (DSC) thermogram of a 50:50 mixture of 2-[2-[4-[(4-chlorophenyl)phenylmethyl]-1-piperazinyl]ethoxy]acetic acid monohydrochloride and 2-[2-[4-[(4-chlorophenyl)phenylmethyl]-1-piperazinyl]ethoxy]acetic acid dihydrochloride.

FIG. 5 is a Differential Scanning Calorimetry (DSC) thermogram of 2-[2-[4-[(4-chlorophenyl)phenylmethyl]-1-piperazinyl]ethoxy]acetic acid dihydrochloride.

FIG. 6 is a ¹³C Nuclear Magnetic Resonance (NMR) spectrum in d₆-DMSO of 2-[2-[4-[(4-chlorophenyl)phenylmethyl]-1-piperazinyl]ethoxy]acetic acid dihydrochloride.

FIG. 7 is an X-Ray Diffraction Pattern (XRD) of 2-[2-[4-[(4-chlorophenyl)phenylmethyl]-1-piperazinyl]ethoxy]acetic acid dihydrochloride.

FIG. 8 is an X-Ray Diffraction Pattern (XRD) of a 50:50 mixture of 2-[2-[4-[(4-chlorophenyl)phenylmethyl]-1-piperazinyl]ethoxy]acetic acid monohydrochloride and 2-[2-[4-[(4-chlorophenyl)phenylmethyl]-1-piperazinyl]ethoxy]acetic acid dihydrochloride.

### DETAILED DESCRIPTION

This invention is directed to an anti-allergenic compound having therapeutic value and a process for its manufacture. In particular, the present invention is directed to the monohydrochloride salt of 2-[2-[4-[(4-chlorophenyl) phenylmethyl]-1-piperazinyl]ethoxy]acetic acid, to compositions containing this compound, and to a process for the preparation of such compound.

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

### Synthesis of [2-[4-[(4-chlorophenyl)phenylmethyl]-1-piperazinyl]ethoxy]acetic acid monohydrochloride

According to one embodiment of the present invention, 2-[2-[4-[(4-chlorophenyl)phenylmethyl]-1-piperazinyl]ethoxy]acetic acid monohydrochloride is prepared according to the following synthetic reaction scheme.

### (a) Reaction of 2-chloroethanol with 4-chlorobenzhydryl piperazine

4-chlorobenzhydryl piperazine (Formula (II)): 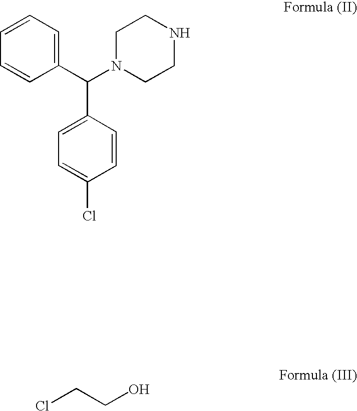
is reacted with a molar excess of 2-chloroethanol (Formula III) in a solvent in the presence of a base, to form 2-[4-[(4-chlorophenyl)phenylmethyl]-1-piperazinyl]ethanol (Formula IV).

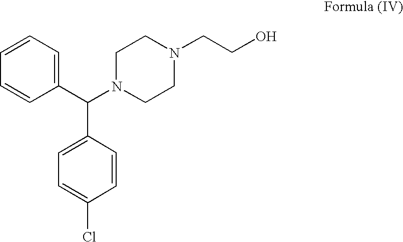

The molar excess of 2-chloroethanol used in this reaction stage is typically between about 1 fold and about 2 fold, preferably about 1.5 fold. Suitable solvents for this synthetic stage include, but are not limited to, aromatic hydrocarbons, such as toluene, xylene, etc., preferably toluene. Suitable bases include, but are not limited to, organic bases, such as suitable acid accepters such as tertiary organic bases, for example, organic amines, such as triethylamine, or inorganic bases, such as sodium carbonate. The base is also typically used in a molar excess of between about 1 and about 2 fold, typically about 1.75 fold, relative to the p-chlorobenzhydryl piperazine. This reaction is typically performed at the reflux temperature of the solvent.

### (b) Reaction of 2-[4-[(4-chlorophenyl)phenylmethyl]-1-piperazinyl]ethanol with a metal haloacetate

2-[4-[(4-chlorophenyl)phenylmethyl]-1-piperazinyl]ethanol (Formula IV) is reacted with a metal haloacetate in the presence of an acid acceptor in a polar solvent to generate 2-[4-[(4-Chlorophenyl)phenylmethyl]-1-piperazinyl]ethoxy]acetic acid (Formula V)

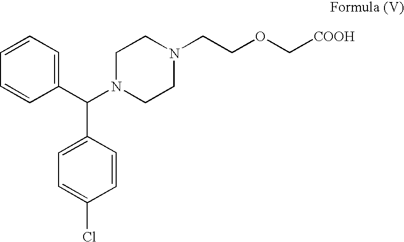

Suitable metal haloacetates include, but are not limited to, sodium, potassium and lithium chloracetates and bromoacetates. A specific metal haloacetate useful in the process of the present invention is sodium chloroacetate. Suitable acid acceptors include, but are not limited to, alkali and alkali-earth metal hydroxides, such as sodium, potassium, lithium, magnesium and calcium hydroxides. Suitable polar solvents include, but are not limited to, dimethylformamide, dimethylacetamide, dimethylsulfoxide, etc. This reaction is typically undertaken at temperatures below room temperature, typically at temperatures between about 0° C. and about 40° C., preferably at a temperature of about 0° C. Reaction times are typically between about 3 and about 8 hours, preferably being between about 4 and about 5 hours.

The molar ratios of metal haloacetate and acid acceptor per mole of the 2-[4-[(4-chlorophenyl)phenylmethyl]-1-piperazinyl]ethanol are typically between about 2 to about 3, and between about 2 to about 3, respectively, preferably being about 2:2.4.

The 2-[4-[(4-chlorophenyl)phenylmethyl]-1-piperazinyl]ethoxy]acetic acid thus prepared was found to be 99% pure by HPLC, and to possess a melting point of 148-150° C., which is higher than the 110 to 115° C. melting point reported in U.S. Pat. No. 4,525,358 for the same material.

### (c) Reaction of 2-[4-[(4-chlorophenyl)phenylmethyl]-1-piperazinyl]ethoxy]acetic acid with hydrogen chloride

2-[4-[(4-chlorophenyl)phenylmethyl]-1-piperazinyl]ethoxy]acetic acid is reacted with hydrogen chloride in a polar solvent, to generate 2-[4-[(4-chlorophenyl)phenylmethyl]-1-piperazinyl]ethoxy]acetic acid monohydrochloride (Formula I).

Suitable polar solvents for this reaction include, but are not limited to, organic solvents such as aliphatic ketones, for example acetone, ethylmethyl ketone, etc. The hydrogen chloride used may be in the form of gaseous anhydrous hydrogen chloride, which is typically bubbled through a solution of the compound of Formula (V), or in the form of an aqueous hydrochloric acid solution. Preferably a 35 to 38% w/w concentration hydrochloric acid solution is used. The molar ratio of HCl to 2-[4-[(4-chlorophenyl)phenylmethyl]-1-piperazinyl]ethoxy]acetic acid is typically between about 1 and about 1.05, and preferably about 1:1.

This reaction is typically carried out at temperatures between about 50° C. and about 100° C., preferably at the reflux temperature of the solvent, with reaction times being between about 8 and about 14 hours, preferably between about 8 and about 10 hours.

### Characterization of 2-[2-[4-[(4-Chlorophenyl)phenylmethyl]-1-piperazinyl]ethoxy]acetic acid monohydrochloride

The monohydrochloride salt of the present invention, 2-[4-[(4-chlorophenyl)phenylmethyl]-1-piperazinyl]ethoxy]acetic acid monohydrochloride, is characterized by DSC, NMR, X-Ray powder diffraction, melting point, elemental analysis, and HPLC. For comparison purposes, certain of these analyses have also been performed for the corresponding dihydrochloride species. DSC analysis was performed using a Perkin Elmer DSC-7 model. X-ray powder Diffraction spectra were recorded on a Regaku XRD Instrument.

DSC Analysis

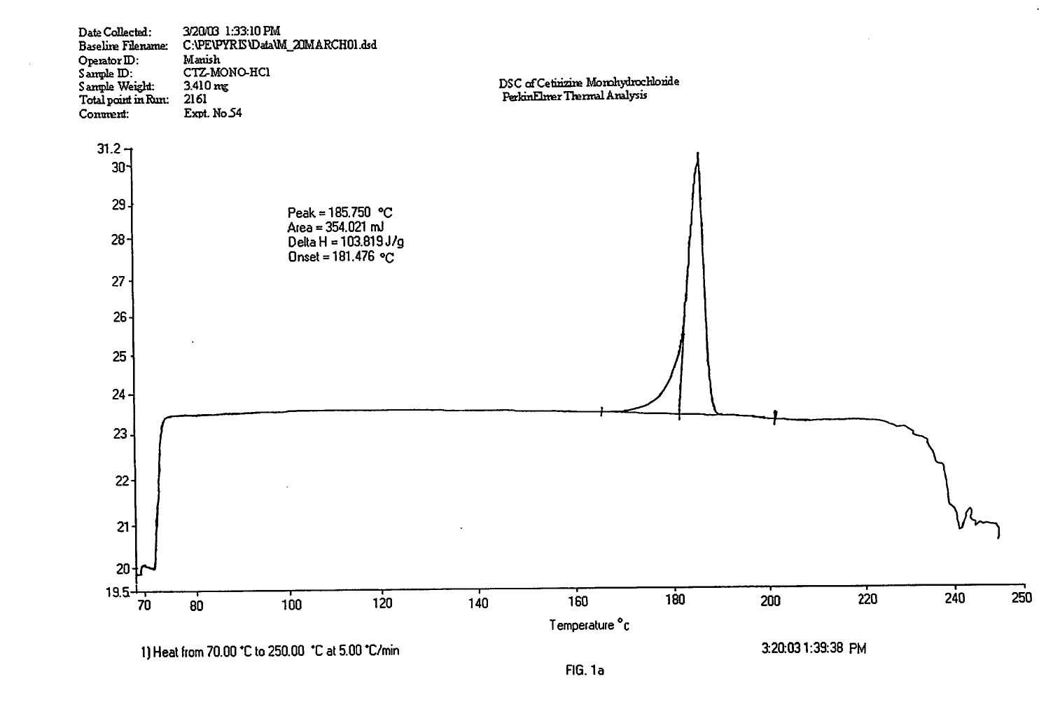

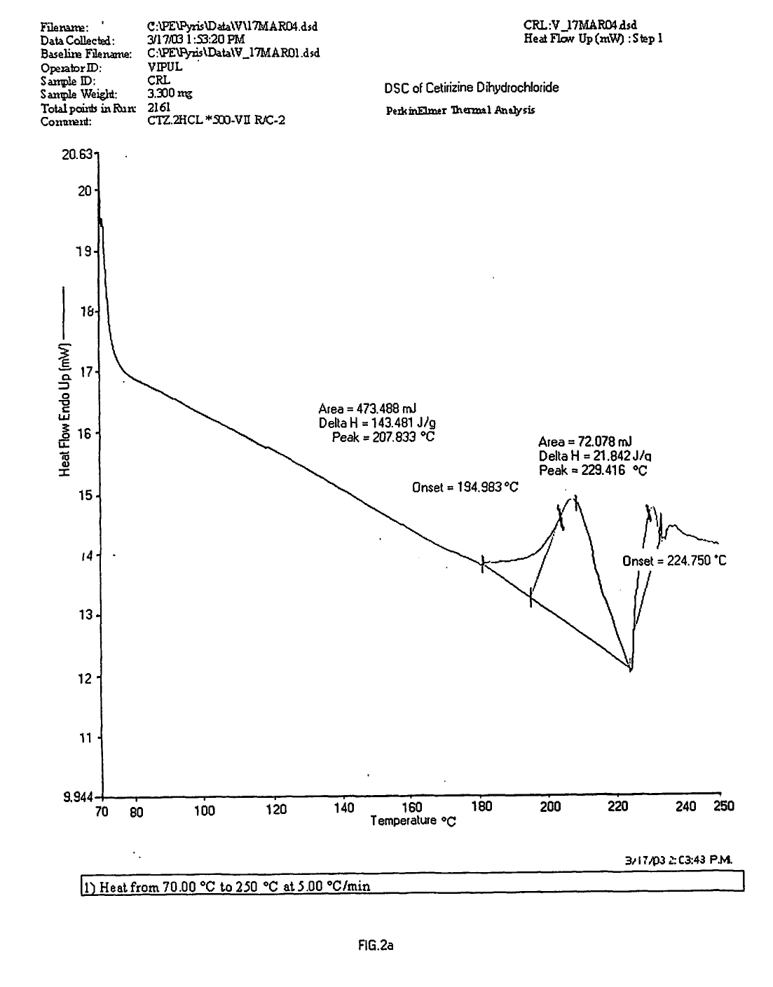

The Differential Scanning Calorimetry (DSC) thermogram of 2-[2-[4-[(4-chlorophenyl)phenylmethyl]-1-piperazinyl]ethoxy]acetic acid monohydrochloride of the invention (FIG. 1) shows a peak endotherm at 185.75° C. As can be seen by a comparison of FIG. 1 with FIG. 5, (which shows the DSC thermogram for the corresponding dihydrochloride salt), this is significantly different than that observed for the dihydrochloride (peak endotherm at 207.83° C.).

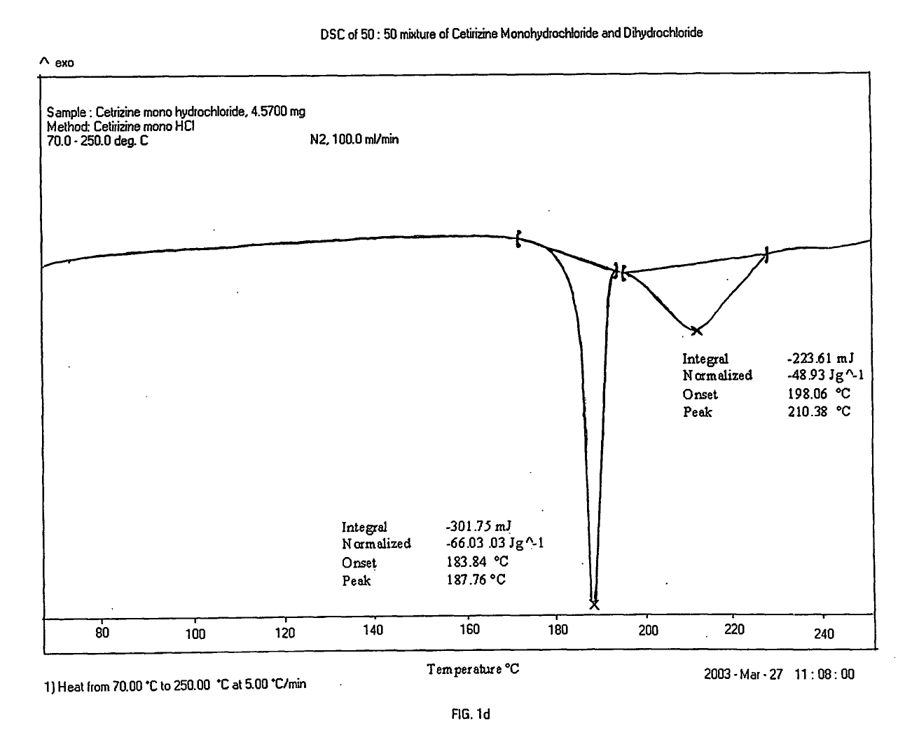

FIG. 4 shows the DSC thermogram of a 50:50 weight percent mixture of the monohydrochloride and dihydrochloride salts. As can be seen, there is a clear differentiation between the peak values for the two salts (187.76° C. for the monohydrochloride and 210.38° C. for the dihydrochloride, respectively).

X-Ray Powder Diffraction

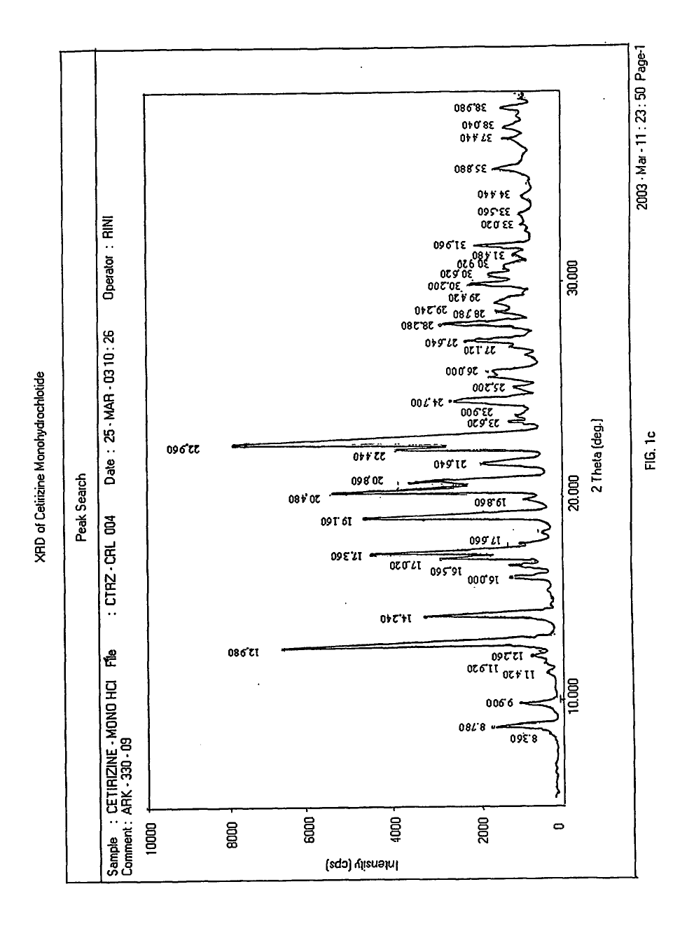

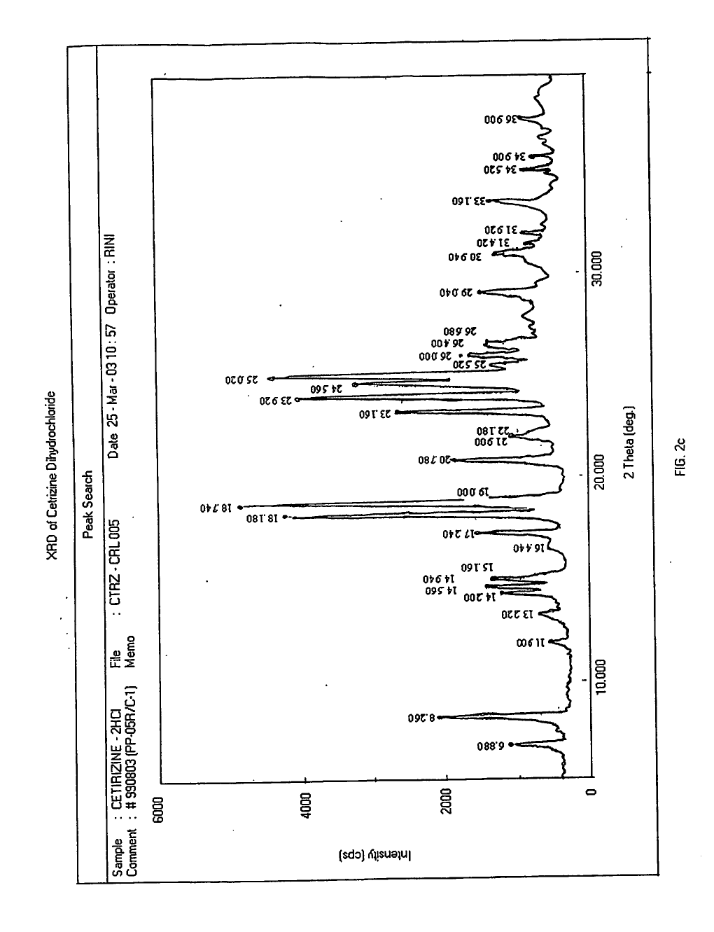

FIGS. 3 and 7 show X-Ray powder diffraction patterns for the monohydrochloride and dihydrochloride salts, respectively. A comparison of the complete diffraction peaks, designated by “2θ” and expressed in degrees, is set forth in Table 1.

TABLE 1
XRD Peaks for the Monohydrochloride and Dihydrochloride
salts of 2-[2-[4-[(4-chlorophenyl)phenylmethyl]-1-
piperazinyl]ethoxy]acetic acid.

| Monohydrochloride Salt | Dihydrochloride Salt |
|---|---|
| 2θ Values (Degrees) | 2θ Values (Degrees) |
| 8.78 | 6.88 |
| 9.90 | 8.26 |
| 12.98 | 11.90 |
| 14.24 | 13.22 |
| 15.00 | 14.22 |
| 16.00 | 14.56 |
| 16.56 | 14.94 |
| 17.02 | 15.16 |
| 17.36 | 17.24 |
| 19.16 | 18.18 |
| 20.48 | 18.74 |
| 20.86 | 19.00 |
| 21.64 | 20.78 |
| 22.44 | 21.90 |
| 22.96 | 23.16 |
| 23.62 | 23.92 |
| 24.70 | 24.56 |
| 25.74 | 25.02 |
| 26.00 | 25.52 |
| 27.46 | 26.00 |
| 28.28 | 26.40 |
| 28.78 | 29.04 |
| 29.24 | 30.94 |
| 30.20 | 31.42 |
| 30.62 | 31.92 |
| 31.96 | 33.16 |
| 35.98 | 34.55 |
| | 34.90 |
| | 36.90 |

A characteristic XRD peak for cetirizine monohydrochloride, designated by “degrees 20”, is 22.96±0.02. This peak is absent in the XRD pattern of cetirizine dihydrochloride. Similarly, a characteristic XRD peak for the dihydrochloride, designated by “degrees 2θ”, is 18.74±0.02. This peak is absent in the XRD pattern of the monohydrochloride.

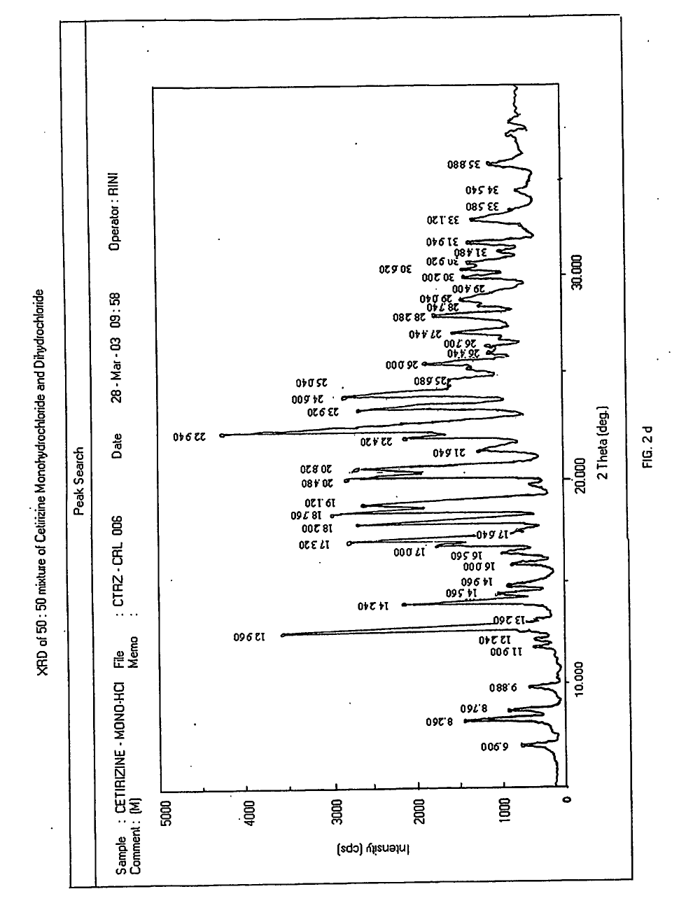

FIG. 8 shows the X-Ray powder diffraction pattern peaks, designated by “2θ” and expressed in degrees, for a 50:50 weight percent mixture of the monohydrochloride and dihydrochloride salts. As can be seen, the two salts are characterized by distinct characteristic peaks.

Elemental Analyses

Elemental analyses for the monohydrochloride and dihydrochloride salts are presented in Table 2.

TABLE 2
Elemental Analyses for the Monohydrochloride and Dihydrochloride
salts of 2-[2-[4-[(4-chlorophenyl)phenylmethyl]-1-
piperazinyl]ethoxy]acetic acid.

| | Monohydrochloride Salt | Dihydrochloride Salt |
|---|---|---|
| Element | Calculated/Found (wt %) | Calculated/Found (wt %) |
| Carbon | 59.25/59.70 | 54.56/54.56 |
| Hydrogen | 6.11/6.56 | 5.84/5.88 |
| Nitrogen | 6.58/6.89 | 6.06/6.21 |
| Chlorine | 8.50/8.35 | 16.67/N.D. |

N.D. = not determined.

NMR Spectroscopy

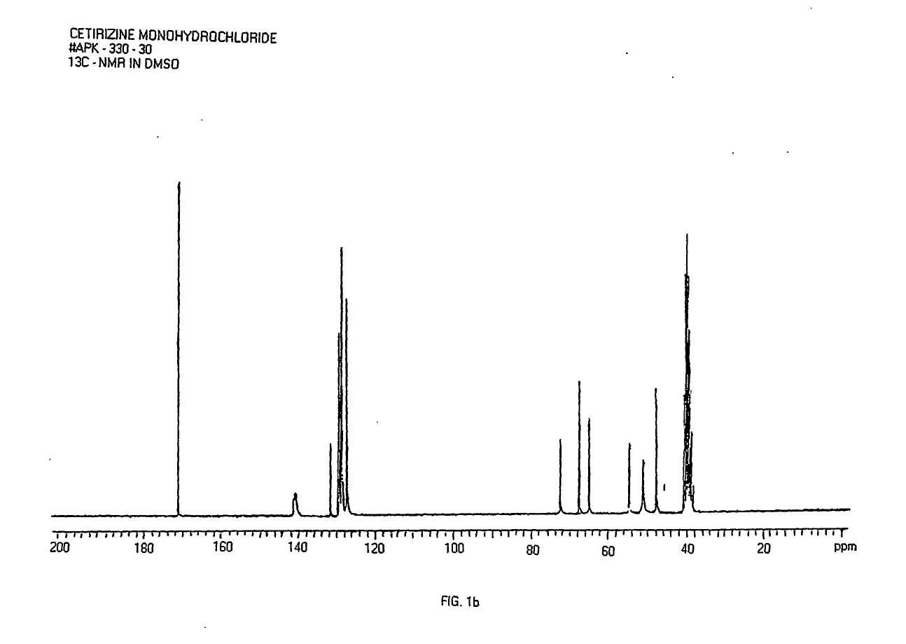

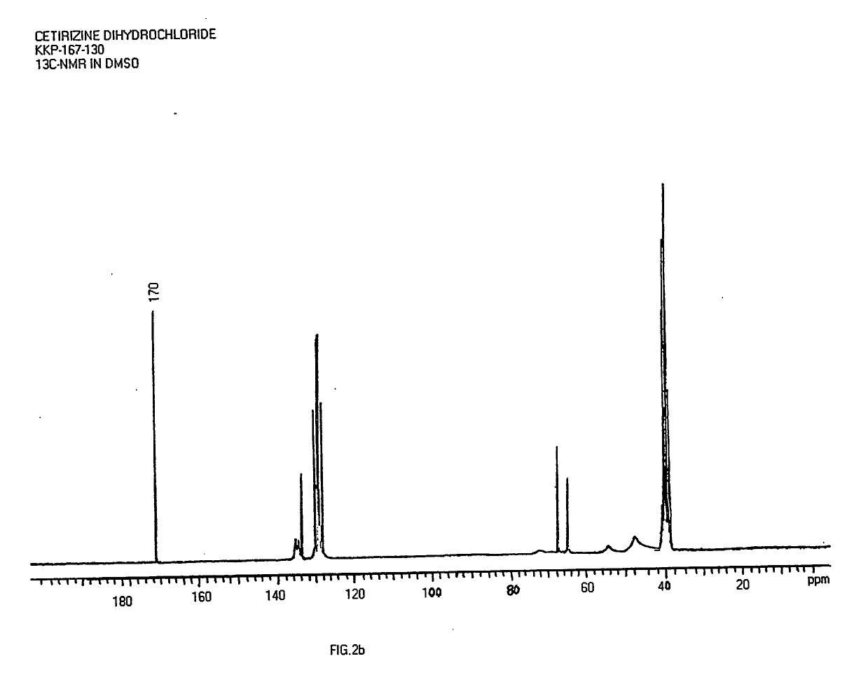

FIG. 2 shows the ¹³C NMR spectrum (in d6-DMSO) for the monohydrochloride of the present invention. For comparison purposes, the ¹³C NMR spectrum (also in d₆-DMSO) for the dihydrochloride is presented in FIG. 6.

The ¹H NMR spectrum for the monohydrochloride compound shows the following peaks (ppm relative to d₆-DMSO, integration values in parentheses): 7.50-7.18 (5H), 4.52 (1H), 4.08 (2H), 3.85-3.72 (2H), 3.43-3.29 (6H), and 3.85-2.50 (4H). Characteristic peaks for the monohydrochloride are at about 4.5 ppm and about 3.2 ppm.

### Therapeutic Compositions and Regimens

The monohydrochloride salt of the present invention can be utilized in the preparation of rapid, controlled and sustained release pharmaceutical formulations, suitable for example, for oral administration. Such formulations may be useful for the treatment of allergic conditions, such as chronic and acute allergic rhinitis, allergic conjunctivitis, pruritus, and urticaria, etc.

The formulations are preferably administered orally, in the form of rapid or controlled release tablets, microparticles, mini tablets, capsules and oral solutions or suspensions, or powders for the preparation thereof. In addition to the monohydrochloride of the present invention as the active substance, oral preparations may optionally include various standard pharmaceutically acceptable carriers, diluents and excipients, such as binders, fillers, buffers, lubricants, glidants, disintegrants, odorants, sweeteners, surfactants and coatings. Some excipients may have multiple roles in the formulations, e.g., act as both binders and disintegrants.

As used herein, the phrase “pharmaceutically acceptable” refers to molecular entities and compositions that are “generally regarded as safe”, e.g., that are physiologically tolerable and do not typically produce an allergic or similar untoward reaction, such as gastric upset, dizziness, doziness and the like, when administered to a human. Preferably, as used herein, the term “pharmaceutically acceptable” means approved by a regulatory agency of the Federal or a state government or listed in the U.S. Pharmacopeia or other generally recognized pharmacopeia for use in animals, and more particularly in humans.

Examples of pharmaceutically acceptable disintegrants for oral formulations useful in the present invention include, but are not limited to, starch, pre-gelatinized starch, sodium starch glycolate, sodium carboxymethylcellulose, croscarmellose sodium, microcrystalline cellulose, alginates, resins, surfactants, effervescent compositions, aqueous aluminum silicates and crosslinked polyvinylpyrrolidone.

Examples of pharmaceutically acceptable binders for oral formulations useful herein include, but are not limited to, acacia; cellulose derivatives, such as methylcellulose, carboxymethylcellulose, hydroxypropylmethylcellulose, hydroxypropylcellulose or hydroxyethylcellulose; gelatin, glucose, dextrose, xylitol, polymethacrylates, polyvinylpyrrolidone, sorbitol, starch, pre-gelatinized starch, tragacanth, xanthane resin, alginates, magnesium-aluminum silicate, polyethylene glycol or bentonite.

Examples of pharmaceutically acceptable fillers for oral formulations include, but are not limited to, lactose, anhydrolactose, lactose monohydrate, sucrose, dextrose, mannitol, sorbitol, starch, cellulose particularly microcrystalline cellulose), dihydro- or anhydro-calcium phosphate, calcium carbonate and calcium sulfate.

Examples of pharmaceutically acceptable lubricants useful in the formulations of the invention include, but are not limited to, magnesium stearate, talc, polyethylene glycol, polymers of ethylene oxide, sodium lauryl sulfate, magnesium lauryl sulfate, sodium oleate, sodium stearyl fumarate, DL-leucine and colloidal silicon dioxide

Examples of suitable pharmaceutically acceptable odorants for the oral formulations include, but are not limited to, synthetic aromas and natural aromatic oils such as extracts of oils, flowers, fruits and combinations thereof. Preferable are vanilla and fruit aromas, including banana, apple, sour cherry, peach and similar aromas. Their use depends on many factors, the most important being the organoleptic acceptability for the population that will be taking the pharmaceutical formulations.

Examples of suitable pharmaceutically acceptable dyes for the oral formulations include, but are not limited to, synthetic and natural dyes such as titanium dioxide, beta-carotene and extracts of grapefruit peel.

Examples of useful pharmaceutically acceptable coatings for the oral formulations, typically used to facilitate swallowing, modify the release properties, improve the appearance, and/or mask the taste of the formulations include, but are not limited to, hydroxypropylmethylcellulose, hydroxypropylcellulose and acrylate-methacrylate copolymers.

Suitable examples of pharmaceutically acceptable sweeteners for the oral formulations include, but are not limited to, aspartame, saccharin, saccharin sodium, sodium cyclamate, xylitol, mannitol, sorbitol, lactose and sucrose.

Suitable examples of pharmaceutically acceptable buffers include, but are not limited to, citric acid, sodium citrate, sodium bicarbonate, dibasic sodium phosphate, magnesium oxide, calcium carbonate and magnesium hydroxide.

Suitable examples of pharmaceutically acceptable surfactants include, but are not limited to, sodium lauryl sulfate and polysorbates.

Examples of suitable pharmaceutically acceptable liquid carriers for orally administrable solutions or suspensions include, but are not limited to, water, alcohols or glycols such as ethanol, isopropanol, propylene glycol, hexylene glycol, glycerol and polyethylene glycol, or mixtures thereof in which the monohydrochloride is dissolved or dispersed, optionally with the addition of non-toxic anionic, cationic or non-ionic surfactants, preservatives, and inorganic or organic buffers.

Suitable examples of pharmaceutically acceptable preservatives include, but are not limited to, various antibacterial and antifungal agents such as solvents, for example ethanol, propylene glycol, benzyl alcohol, chlorobutanol, quaternary ammonium salts, and parabens (such as methyl paraben, ethyl paraben, propyl paraben, etc.).

The therapeutically acceptable quantity of the monohydrochloride salt administered is an anti-allergic effective amount, which varies, dependent on the selected compound, the mode of administration, treatment conditions, age and status of the human or animal patient, and is subject to the final decision of the physician, clinician or veterinary doctor monitoring the course of treatment.

An anti-allergic effective amount means an amount sufficient to prevent or reduce the symptoms of an allergic reaction or syndrome. Suitable oral and parenteral doses may vary within the range from about 1 mg to about 25 mg, preferably between about 2.5 mg to about 20 mg, more preferably between about 5 mg to about 10 mg. The monohydrochloride may be formulated in a single dosage form that contains a dose range wherein the monohydrochloride salt is present in a range from about 1 to about 40% w/w of the weight of the formulated product, preferably from about 2.5 mg to about 20 mg, and more desirably from about 5 to about 10 mg of the active substance per unit dose.

A constant supply of the therapeutic compound can be ensured by providing a therapeutically effective dose (i.e., a dose effective to induce metabolic changes in a subject) at the necessary intervals, e.g., daily, every 12 hours, etc. These parameters will depend on the severity of the allergic condition being treated, the regimen of any other drugs being administered, other actions, such as diet modification, that are implemented, the weight, age, and sex of the subject, and other criteria, which can be readily determined according to standard good medical practice by those of skill in the art.

A subject in whom administration of the monohydrochloride of the invention is an effective antiallergenic regimen for a disease or disorder is preferably a human, but can be any animal, including a laboratory animal in the context of a clinical trial or screening or activity experiment. Thus, as can be readily appreciated by one of ordinary skill in the art, the methods and compositions of the present invention are particularly suited to administration to any animal, particularly a mammal, and including, but by no means limited to, domestic animals, such as feline or canine subjects, farm animals, such as but not limited to bovine, equine, caprine, ovine, and porcine subjects, wild animals (whether in the wild or in a zoological garden), research animals, such as nice, rats, rabbits, goats, sheep, pigs, dogs, cats, etc., avian species, such as chickens, turkeys, songbirds, etc., i.e., for veterinary medical use.

### EXAMPLES

The following Example illustrates the invention, but is not limiting thereof.

Materials

The dihydrochloride salt (2-[2-[4-[(4-chlorophenyl)phenylmethyl]-1-piperazinyl]ethoxy]acetic acid dihydrochloride) may be prepared according to the process set forth in U.S. Pat. No. 4,535,358.

### Preparation of Cetirizine Monohydrochloride

### (a) Synthesis of Chloroethanol Adduct (2-[4-[(4-chlorophenyl)phenylmethyl]-1-piperazinyl]ethanol)

A mixture of 4-chlorobenzhydryl piperazine (100 g, 0.348 mol), 2-chloroethanol (41.8 g, 0.519 mol) and triethylamine (61.8 g, 0.61 mol) in toluene (470 ml) was heated to reflux. The reaction mixture was then cooled to room temperature and washed with water (260 ml×3). The toluene was evaporated using a rota vapour under reduced pressure to dryness to give a viscous oil (115 g). The 1-[(4-chlorophenyl)phenylmethyl]piperazinyl]ethanol thus prepared had a boiling point of 220° C. at 0.065 mbar, and a 93% purity, by HPLC.

### (b) Synthesis of Cetirizine Base (2-[2-[4-[(4-chlorophenyl)phenylmethyl]-1-piperazinyl]ethoxy]acetic acid)

(i) To a solution of 1-[[(4-chlorophenyl)phenylmethyl]piperazinyl]ethanol (100 g, 0.302 mol) in 326 ml of dimethyl formamide, potassium hydroxide pellets (40.84 g, 0.728 mol) were added followed by sodium chloroacetate (70.5 g, 0.6052 mol) in fractions. The reaction mixture was well stirred at 153° C. for 4-5 hours. 1.26 liter distilled water was then added to the reaction mixture and the pH was adjusted to between 4.0 to 4.5 using 50% aqueous HCl solution. The crude reaction mixture was then extracted twice with dichloromethane (300 ml×2). The organic layers were combined, washed with water and brine and dried over magnesium sulfate. Dichloromethane was evaporated under reduced pressure to give 130 g of thick syrup of 2-[2-[4-[(4-Chlorophenyl)phenylmethyl]-1-piperazinyl]ethoxy]acetic acid (cetirizine base). To the crude syrup, 1.5 liter of toluene was added and mixed. The toluene was then distilled off under vacuum to generate highly viscous syrup. To the crude reaction mixture, n-hexane (800 ml) was added, stirred for half an hour, and filtered. After drying at 60-65° C. for 5 hours under vacuum 100 g (99% HPLC purity assessment) of 2-[2-[4-[(4-chlorophenyl)phenylmethyl]-1-piperazinyl]ethoxy]acetic acid was obtained, with a melting point of 148° C.-150° C.

(ii) Alternatively, the cetirizine base was prepared by adding 130 g of 2-[2-[4-[(4-chlorophenyl)phenylmethyl)-1-piperazinyl]ethoxy]acetic acid dihydrochloride to 400 ml of distilled water and adjusting the pH of the solution to 4.7 using 25% W/V sodium hydroxide solution. The mixture was then warmed to 50 to 55° C. for 4 hours. Before cooling to room temperature, it was extracted with chloroform (4×400 ml). The combined chloroform extracts were washed with distilled water (5×400 ml) and then evaporated to dryness under vacuum to obtain a thick viscous mass. Toluene (750 ml) was added to the viscous mass. It was stirred well and the toluene was distilled off under vacuum to obtain a thick mass of the cetirizine base. The mixture was cooled to room temperature and then 500 ml hexane was added. After stirring for half an hour, the mixture was filtered. The filtered mass was dried under vacuum at 50 to 60° C. for 5 hours. Yield was 50 g and HPLC purity was 98.99%. Chlorine content was below 0.05%, and residue on ignition confirmed a chlorine content below 0.1%. The melting point of this product was observed to be between 148° C. to 150° C.

### (c) Synthesis of Cetirizine Monohydrochloride (2-[2-[4-[(4-chlorophenyl)phenylmethyl]-1-piperazinyl]ethoxy]acetic acid monohydrochloride)

25 g (0.64 mol) of cetirizine base as prepared in (b)(i) was added to 250 ml of anhydrous acetone, and to the resulting mixture 2.395 g (0.065 mol) of aqueous HCl was carefully added. The mixture was then refluxed for 8 h, cooled to room temperature and filtered. The wet cake was washed with 50 ml of cold acetone and dried under vacuum at 60-65° C. for 5 hours to yield 25 g of cetirizine monohydrochloride. HPLC purity of the monohydrochloride salt was found to be 99.61%. The observed melting point was 186° C.-188° C. In DSC analysis, a sharp signal at 187.9° C. was observed, and the product had 8.5% chloride content upon elemental analysis.

The present invention is not to be limited in scope by the specific embodiment described herein. Various modifications of the invention in addition to those described herein will become apparent to those skilled in the art from the foregoing description. Such modifications are intended to fall within the scope of the appended claims.

All patents, applications, publications, test methods, literature, and other materials cited herein are hereby incorporated by reference in their entirety.

We claim:

1. 2-[2-[4-[(4-chlorophenyl)phenylmethyl]-1-piperazinyl]ethoxy]acetic acid monohydrochloride.

2. The product of claim 1, in substantially crystalline form.

3. The product of claim 1, having a melting point between about 186° C. and about 188° C.

4. The product of claim 1, having characteristic ¹H nuclear magnetic resonance peaks in DMSO at about 4.5 ppm and at about 3.2 ppm.

5. The product of claim 1, having a characteristic X-ray diffraction pattern 2θ peak at about 22.96±0.02 degrees.

6. The product of claim 1, having a DSC thermogram peak at about 185.75° C.

7. The product of claim 1, having a chlorine content of about 8.5 percent by weight.

8. A process for the preparation of 2-[2-[4-[(4-chlorophenyl)phenylmethyl]-1-piperazinyl]ethoxy]acetic acid monohydrochloride, comprising:
(a) contacting a compound having the formula 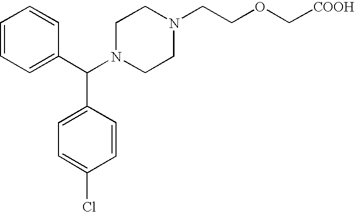
with an aqueous hydrochloric acid solution in a solvent for a sufficient contact time to form 2-[2-[4-[(4-chlorophenyl)phenylmethyl]-1-piperazinyl]ethoxy]acetic acid monohydrochloride; and
(b) isolating the 2-[2-[4-[(4-chlorophenyl)phenylmethyl]-1-piperazinyl]ethoxy]acetic acid monohydrochloride.

9. The process of claim 8, wherein the solvent is an organic solvent

10. The process of claim 9, wherein the solvent is a polar solvent.

11. The process of claim 10, wherein the solvent is acetone.

12. The process of claim 8, wherein the contacting step is conducted at a temperature above the reflux temperature of the solvent.

13. The process of claim 8, wherein the contact time is between about 6 hours and about 10 hours.

14. The process of claim 8, wherein the molar ratio of 2-[2-[4-[(4-chlorophenyl)phenylmethyl]-1-piperazinyl]ethoxy]acetic acid to hydrochloric acid in step (a) is about 1:1.

15. The process of claim 9, wherein the 2-[2-[4-[(4-chlorophenyl)phenylmethyl]-1-piperazinyl]ethoxy]acetic acid monohydrochloride is isolated in greater than about 98% purity.

16. A process for the preparation of 2-[2-[4-[(4-chlorophenyl)phenylmethyl]-1-piperazinyl]ethoxy]acetic acid monohydrochloride, comprising:
(a) reacting chloroethanol with p-chlorobenzhydryl piperazine to form an adduct thereof;
(b) converting the adduct from step (a) to 2-[2-[4-[(4-chlorophenyl)phenylmethyl)-1-piperazinyl]ethoxy]acetic acid; and
(c) converting the product from step (b) to 2-[2-[4-[(4-chlorophenyl)phenylmethyl]-1-piperazinyl]ethoxy]acetic acid monohydrochloride.

17. The process of claim 16, wherein step (b) comprises contacting 2-[4-[(4-chlorophenyl)phenylmethyl]-1-piperazinyl]ethanol with sodium chloroacetate in the presence of an alkali metal hydroxide to form 2-[2-[4-[(4-chlorophenyl)phenylmethyl]-1-piperazinyl]ethoxy]acetic acid.

18. The process of claim 16, wherein step (c) comprises contacting 2-[2-[4-[(4-chlorophenyl)phenylmethyl]-1piperazinyl]ethoxy]acetic acid with an aqueous hydrochloric acid solution to form 2-[2-[4-[(4-chlorophenyl)phenylmethyl]-1-piperazinyl]ethoxy]acetic acid monohydrochloride.

19. A composition comprising an anti-allergic effective amount of 2-[2-[4-[(4-chlorophenyl)phenylmethyl]-1-piperazinyl]ethoxy]acetic acid monohydrochloride and a pharmaceutically acceptable carrier or excipient.
